(19) United States
(12) Patent Application Publication
(10) Pub. No.: US 2007/0225756 A1
(43) Pub. Date: Sep. 27, 2007

(54) Closure device and insertion assembly

(75) Inventors: Fredrik Preinitz, Uppsala (SE); Per Egnelov, Phuket (TH)

Correspondence Address: FOLEY AND LARDNER LLP;SUITE 500, 3000 K STREET NW, WASHINGTON, DC, 20007, US

(73) Assignee: RADI MEDICAL SYSTEMS AB

(21) Appl. No.: 11/386,082

(22) Filed: Mar. 22, 2006

(51) Int. Cl. A61B 17/08

(52) U.S. Cl. 606/213

(57) ABSTRACT

A medical apparatus (100) is provided, comprising a closure device (102), which comprises a locking member (130) and a tubular member (121) having a distal set of struts (122) and a proximal set of struts (123), said struts being provided with a hinge section, and an insertion assembly (101), which comprises a holder (106) and an actuator (107), the holder being releasably engaged in the locking member and the actuator being releasably engaged with the tubular member, wherein, in response to a relative movement between the holder and the actuator, the closure device is movable between a first elongated tubular introduction configuration and a second positioning configuration in which the tubular member is compressed such that the distal and proximal sets of struts have moved radially away from a longitudinal central axis of the closure device.

### FIELD OF THE INVENTION

The present invention relates generally to a medical apparatus for closing an opening or defect in an organ within a living body, e.g. a septal defect in a heart or a percutaneous puncture in a vessel wall (such as walls in arteries, or other blood vessels), and in particular to an expandable and repositionable closure device, which can be remotely maneuvered from an initial positioning configuration to a final configuration in which the opening or defect is closed, and more particularly to a mechanical actuator by which the expandable and repositionable closure device is inserted, positioned and delivered in the opening or defect.

### BACKGROUND OF THE INVENTION

The closing of an opening in an organ of a patient is a medical procedure that frequently has to be practised by doctors or other trained medical personnel. The opening may be a hole created by the doctor for a specific and usually temporary purpose, or the opening can be a congenital or acquired defect. An example of the former would be a puncture hole created in a patient's femoral artery to obtain access to the coronary system, while an example of the latter is a septal defect in a patient's heart. For descriptive and illustrative purposes the present invention will be described with reference to such a septal defect, although such techniques can be applied to other fields of application.

As is well-known, the human heart is divided into four chambers: the left atrium, the right atrium, the left ventricle, and the right ventricle. The atria are separated from each other by the interatrial septum, and the ventricles are separated by the interventricular septum.

Either congenitally or by acquisition, abnormal openings or holes can form between the chambers of the heart, causing shunting of blood through the opening or hole. For example, with an atrial septal defect, blood is shunted from the left atrium to the right atrium, which produces an overload of the right side of the heart. In addition to left-to-right shunts such as occur in patent ductus arteriosus from the aorta to the pulmonary artery, the left side of the heart has to work harder because some of the blood will recirculate through the lungs instead of going to the rest of the body. The ill effects of such lesions usually cause added strain on the heart with ultimate failure if not corrected.

One way to cure a septal defect in the septum of a heart is to position and anchor a specially designed closure device at the septum such that both sides of the septal defect are spanned by the closure device to thereby close the defect. Examples of such septal defect closure devices are known from the U.S. Pat. Nos. 5,853,422; 6,024,756; 6,117,159 and 6,312,446 to Huebsch et al., which disclose a closure device comprising a cylindrical shaft of metal or polymeric material with concentric parallel cuts through the wall of the device to thereby create flattened support struts. The centers of the support struts are intended to move radially away from the longitudinal axis of the device in a hinge like fashion in response to movements of the proximal and distal ends of the device towards the centre thereof. The patents show further a number of different deployment catheters by which the closure device can be positioned and delivered. The deployment catheters are, however, described in a rather rudimentary fashion, and do not seem to include all the members necessary to effect the movements and functions of the closure device.

A similar septal defect closure device is also disclosed in the international application WO 2005/006990 A2.

### SUMMARY OF THE INVENTION

Within the medical field it is of utmost importance that closure apparatuses work properly, and a general object of the present invention is therefore to improve a medical apparatus comprising a closure device of the aforementioned type and an accompanying mechanical actuator in such a way that a safe and user-friendly medical apparatus is obtained, wherein the movements of the closure device can be controlled in a reliable way by the mechanical actuator.

According to the present invention, a medical closure apparatus comprises a closure device which, in turn, comprises an elongated tubular member in which a first set of longitudinal slits or cuts has been made on a first side of a shorter uncut central portion and a second set of longitudinal slits or cuts has been made on the opposite side of the central portion. On each side of the central portion, the slits extend towards the ends of the tubular member to terminate a short distance before the respective end, such that uncut proximal and distal end portions are formed. The tubular member, which is made from a flexible and preferably resorbable material, has thereby been provided with proximal and distal sets of struts or ribs. The distal ends of the distal struts are flexibly connected to the distal end portion of the tubular member, while the proximal ends of the distal struts are flexibly connected to the central portion. Similarly, the proximal ends of the proximal struts are flexibly connected to the proximal end portion of the tubular member, while the distal ends of the proximal struts are flexibly connected to the central portion. The struts are further each provided with a weakened section, which can act as a hinge, such that each strut in effect is divided into two articulated arms.

When the closure device during use is compressed such that the distal and proximal end portions are forced towards each other, the weakened sections of the struts move radially out from the longitudinal central axis of the closure device, and the respective arms of the struts assume an essentially perpendicular angle to the central axis of the closure device. The closure device comprises further a central cylindrical locking member, which preferably is separate from the tubular member and which over its length comprises several portions with different diameters. In use, the cylindrical locking member is inserted into the tubular member such that the distal end portion of the tubular member abuts a distal end rim of the locking member, and the proximal end portion of the tubular member is then pushed over a proximal end rim of the locking member. In the compressed state, the central, proximal and distal portions of the tubular member fit snugly over respective portions of the central locking member, and the closure device is held in the compressed state by the enlarged distal and proximal rim portions of the locking member, which prevents the closure device from resuming its original elongated shape.

In accordance with the present invention, the medical closure apparatus comprises further a mechanical actuator by which the closure device can be maneuvered through four (4) well-defined configurations: an introduction configuration, a positioning configuration, a closed configuration, and a locked configuration. The mechanical actuator comprises an actuating member and a holder, whose relative longitudinal translational motion causes the closure device to transform from the introduction configuration via the positioning configuration to the closed configuration, and—if desired—back to the introduction configuration. In a preferred embodiment of the present invention, the actuating member comprises a pusher and a retractor, which move together as a unit to accomplish the first three configurations of the closure device, but which are moved in relation to each other to accomplish the final locked configuration of the closure device. The medical closure apparatus comprises further a catheter, inside which the mechanical actuator can slide. If the closure apparatus is to be used to close a puncture hole in, for example, a femoral artery, the catheter is preferably replaced with an introducer, which normally has been used during a previous medical procedure and which already is in place in the artery.

### BRIEF DESCRIPTION OF THE DRAWINGS

FIG. 1 is a schematic illustration of a human heart having an atrial as well as a ventricular septal defect.

FIG. 2 is a schematic illustration of a human heart having a septal defect, which is to be closed by means of a medical procedure that, in a first step, involves the introduction of a septal defect closure device according to the present invention.

FIG. 3 illustrates an intermediate step in the medical procedure, in which a distal portion of the closure device of FIG. 2 is expanded in order to locate the septal defect from the distal side of the septal defect.

FIG. 4 illustrates the closure device of FIG. 2, which has been positioned in the septum to close the septal defect therein.

FIG. 5 shows a septal defect closure device according to the present invention in an introduction configuration before any longitudinal compression of the closure device.

FIG. 6 shows the closure device of FIG. 5 in an intermediate semi-compressed positioning configuration.

FIG. 7 shows the closure device of FIG. 5 in another intermediate semi-compressed positioning configuration.

FIG. 8 shows a locking member, which according to one embodiment constitutes a separate part of a septal defect closure device.

FIG. 9 shows the closure device of FIG. 5 in a closed configuration.

FIG. 10 shows the closure device of FIG. 5 in another closed configuration, in which distal portions of a mechanical actuator are visible.

FIG. 11 illustrates the closure device of FIG. 5 in a final locked configuration.

FIG. 12 is a sectional drawing of a medical apparatus according to the present invention shown in an introduction stage of a closure delivering operation.

FIG. 13 is a sectional drawing of the medical apparatus of FIG. 12 shown in a positioning stage of the closure delivering operation.

FIG. 14 is a sectional drawing of the medical apparatus of FIG. 12 shown in a closing stage of the closure delivering operation.

FIG. 15 is a sectional drawing of the medical apparatus of FIG. 12 shown in a locking stage of the closure delivering operation.

FIG. 16 is a sectional drawing of the medical apparatus of FIG. 12 shown in a releasing stage of the closure delivering operation.

### DETAILED DESCRIPTION OF PREFERRED EMBODIMENTS OF THE INVENTION

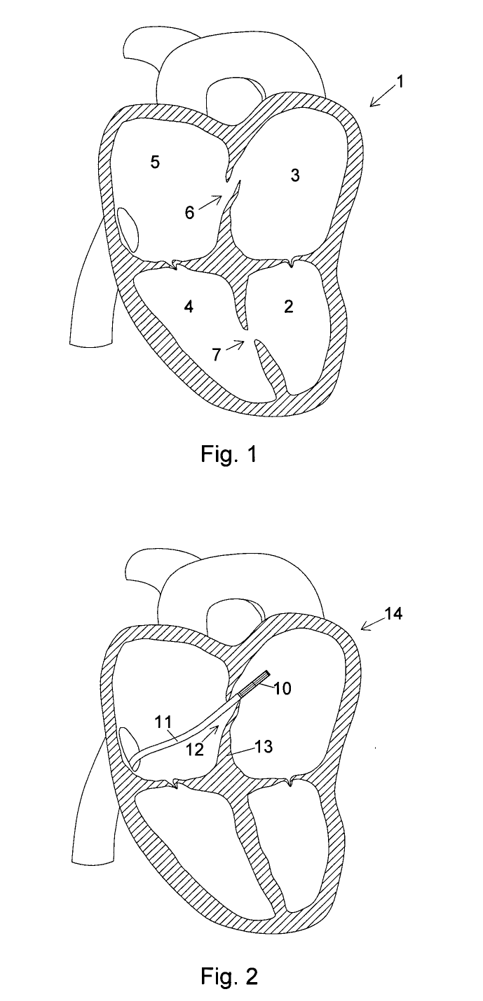

A schematic cross-sectional view of a human heart 1 is shown in FIG. 1. The heart 1, with its left ventricle 2, left atrium 3, right ventricle 4, and right atrium 5, suffers from an atrial septal defect 6 as well as a ventricular septal defect 7. Below a medical procedure will be discussed in which an atrial septal defect is closed. It should, however, be clear that a septal defect closure device according to the present invention equally well could be employed to close a ventricular septal defect like ventricular septal defect 7 of FIG. 1. It should further be noticed that the septal defects 6, 7 can be accessed from different vessels, e.g. from the superior or inferior vena cava, or from the aorta. This means, in turn, that throughout the present description terms like “distal” and “proximal” should always be seen from the end of a delivering catheter, through which a septal defect closure device is delivered (and not from any particular chamber or vessel of a heart).

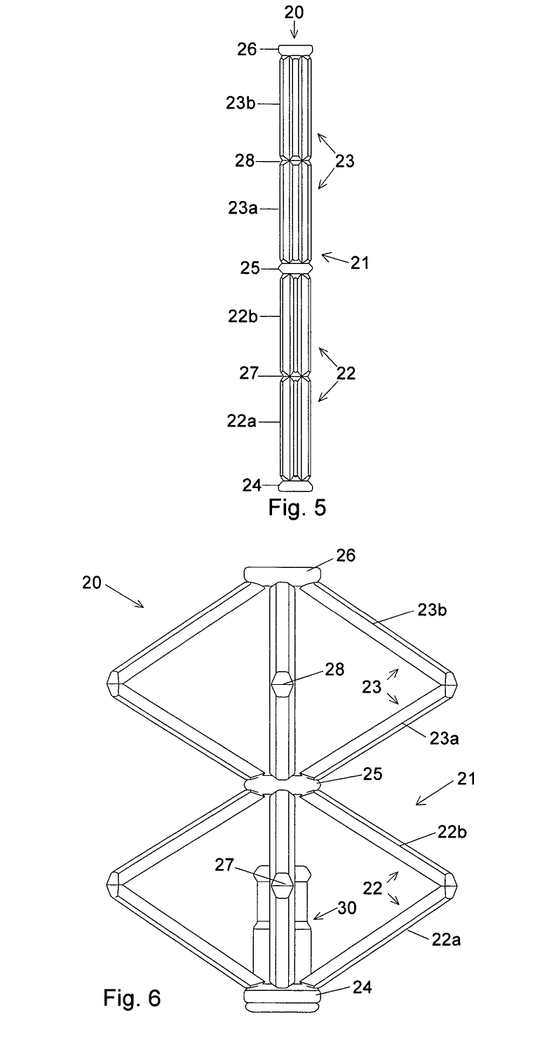

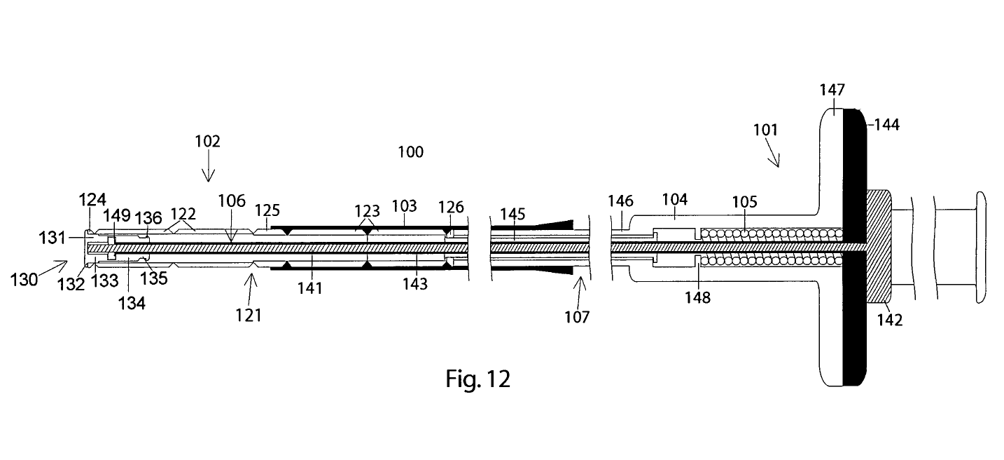

In conjunction with FIGS. 2 to 4, a medical procedure will be briefly described, in which a medical apparatus comprising a septal defect closure device is employed to close a septal defect in the septum of a heart; and thereafter different parts and functions of the closure device will be described in detail in conjunction with FIGS. 5 to 11. The medical apparatus comprises further a mechanical actuator whose operation is described with respect to FIGS. 12 to 16.

FIG. 2 illustrates a septal defect closure device 10, which by use of a mechanical actuator (not shown in FIG. 2) has been delivered through a delivering catheter 11 and introduced into an atrial septal defect 12 in the atrial septum 13 of a heart 14. The closure device 10 is of the same general construction that has been generally described above, and comprises an elongated tubular member in which distal and proximal sets of struts have been provided. The distal struts extend from a central portion of the closure device 10 to a distal end portion thereof, and the proximal struts extend from a proximal end portion of the closure device 10 to the central portion. As already discussed, each strut is provided with a thinner and thereby weaker section that can act as a hinge, and each strut is thereby effectively divided into two hinge-connected arms. In FIG. 2, the closure device 10 is shown in an initial introduction configuration, in which the arms of each strut are substantially aligned with each other. In this introduction configuration, the closure device 10 has therefore a generally elongated tubular shape, which facilitates the introduction of the closure device 10 into the artery and heart of a patient. The introduction configuration is defined as the configuration that the closure device assumes by itself, i.e. without any compression being induced by a mechanical actuator (not shown in FIG. 2) connected to the closure device. In this introduction configuration, the closure device has therefore a generally tubular shape, although the closure device could be preformed such that the arms of each strut exhibit a small positive angle in relation to each other. Such a positive angle guarantees the proper radial expansion of the tubular member during longitudinal compression of the tubular member.

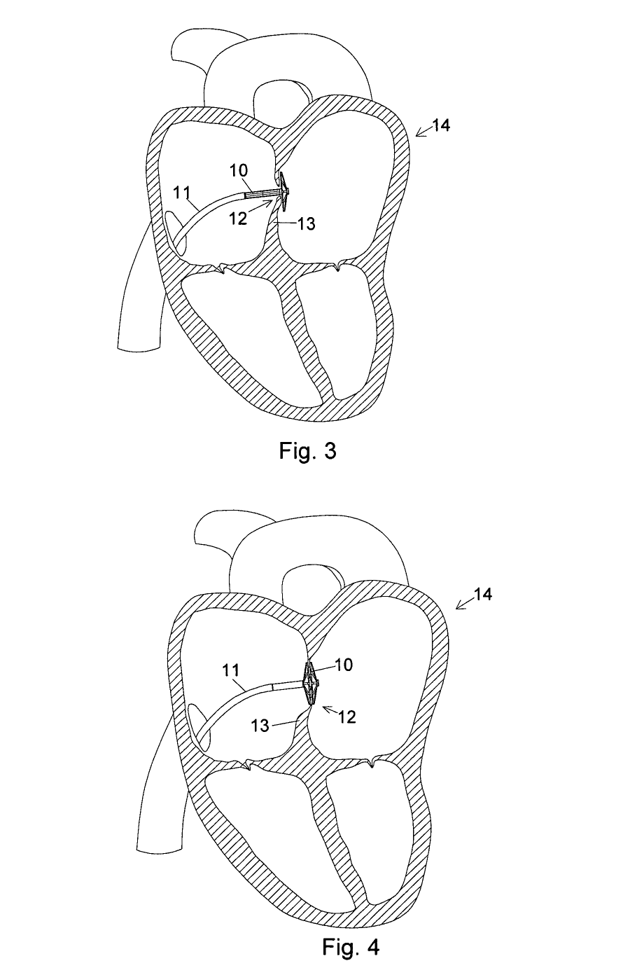

To ascertain correct positioning of the closure device 10 with respect to the septal defect 12, the distal set of struts can be moved radially outwards from the central axis of the closure device 10, such that a partly expanded configuration is obtained. The radial movements of the distal struts are effectuated by partially compressing the closure device 10 through the maneuvering of a mechanical actuator (not shown in FIGS. 2-4). In this semi-expanded locating or positioning configuration, the closure device 10 is retracted until the distal struts abut the distal side of the atrial septum 13 surrounding the septal defect 12. The septal defect 12 can thereby be located by a doctor, who in this phase of the medical procedure will feel a marked increase in resistance against further retraction. This intermediate step of the medical procedure is depicted in FIG. 3. The function and operation of the mechanical actuator that effectuates the semi-expanded configuration shown in FIG. 3 are thoroughly explained below.

When the atrial septum 13 and thereby the septal defect 12 have been correctly located, the closure device 10 is fully expanded such that the proximal struts as well as the distal struts are forced radially outwards by maneuvering of the mechanical actuator mentioned above. In this septal defect closing configuration, the closure device 10 spans both the distal side and the proximal side of the septal defect 12. As can be seen in FIG. 4, the closure device 10 sandwiches the atrial septum 13 to thereby close the septal defect 12 therein. It can be mentioned that the term “close” or similar terms used herein in conjunction with the description of the closing of a septal defect should not be taken too literally. Such terms are meant to encompass all stages from actually sealing or closing off a septal defect to merely restricting the flow of blood therethrough, the important thing being that the closure device permits and facilitates healing of the septal (or other type of) defect over time. To improve the sealing capability of a closure device of the present type, it is possible that the distal and/or proximal struts at least partly are covered by a thin membrane, or formed integrally with a thin membrane, which preferably is made from a resorbable material. This feature may in particular be advantageous when the closure device is used to seal a puncture hole in a vessel wall.

A special feature of the closed configuration illustrated in FIG. 4 is that the closure device 10 still is repositionable. This means that by use of a mechanical actuator (not shown in FIG. 4), the closure device 10 is reversibly movable between the configurations described above in conjunction with FIGS. 2-4, i.e. from the closed configuration of FIG. 4, to the intermediate positioning configuration of FIG. 3, and back to the original introduction configuration of FIG. 2. The closure device 10 can then be retracted out of the patient's body and be disposed, or can once again be positioned by repeating the steps illustrated above. The closed configuration of the closure device 10 is defined as the extreme end position of the different and gradually changing positioning configurations. In the closed configuration essentially no further compression of the closure device 10 is possible while still having a reversibly movable closure device 10. The latter will be further discussed below. In accordance with the present invention, a closure device encompasses a fourth configuration, in which the closure device is irreversibly locked. The transition from the closed configuration to this locked configuration is effectuated by the mechanical actuator mentioned above. A special feature of the present closure device is that a doctor will feel when the closed configuration has been reached, so that he or she can decide whether the mechanical actuator should be maneuvered such that the final locked configuration is achieved. Having in mind that the closed configuration constitutes a situation from which the closure device can be removed, whereas the locked configuration implies a non-retrievable closure device, the importance of having a well-defined transition between these two states should be appreciated. Also this feature and how the locked configuration is achieved by a mechanical actuator will be further discussed below.

An embodiment of a septal defect closure device 20 according to the present invention is illustrated in FIG. 5. FIG. 5 shows the closure device 20 in a first or introduction configuration in which the closure device 20 has the general shape of an elongated tubular member 21, through which a number of longitudinal, parallel cuts or slits have been made to thereby form a first or distal set of struts 22 and a second or proximal set of struts 23. The first strut set 22 extends between a first end portion 24 of the tubular member 21 and a central portion 25 thereof, while the second strut set 23 extends between the central portion 25 and a second end portion 26 of the tubular member 21. The first and second end portions 24, 26 as well as the central portion 25 are uncut and are shorter than the slit portions of the tubular member 21. Somewhere along the length of the first set of struts 22, the tubular member 21 has been provided with a circumferential weakened section 27 in that material has been removed (or weakened in another fashion) from this ring-shaped section of the tubular member 21. The weakened thinner section 27 of each strut 22 will thereby act as a hinge or articulation 27, which effectively divides each strut 22 into two articulated arms: a first or distal arm 22a and a second or proximal arm 22b. Similarly, the struts in the second set of struts 23 are each provided with a hinge section 28, which in effect divides each strut 23 into two articulated arms: a first or distal arm 23a and a second or proximal arm 23b.

Here it should be emphasized that the term “tubular” is merely intended to indicate the general shape of an elongated, cylindrical member, which comprises a number of struts, the ends of which are connected to shorter ring-shaped members, and which in a first introduction configuration assumes a tubular shape. In other words, a tubular member, like tubular member 21, does not actually have to be cut or slit in order to create distal and proximal struts. On the contrary, a tubular member, having struts with weakened hinge-sections as well as ring-shaped central, distal and proximal end portions, can advantageously be directly produced in this form, e.g. by injection molding. Furthermore, the struts of a tubular member, like tubular member 21, do not have to be exactly aligned with each other. Instead, a tubular member can be preformed in such a way that the two arms of a strut exhibit an angled relation to each other, to thereby guarantee that the arms actually bend outwards during compression of the tubular member. Nevertheless, the definition of the introduction configuration is still the configuration or state wherein a closure device has not been subjected to any compression by use of a mechanical actuator. The introduction configuration may therefore also be regarded as the “natural” state of the closure device.

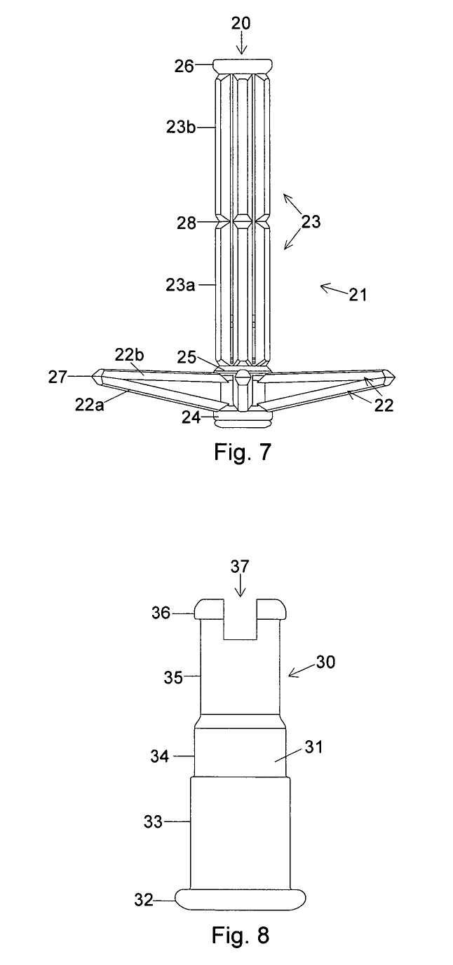

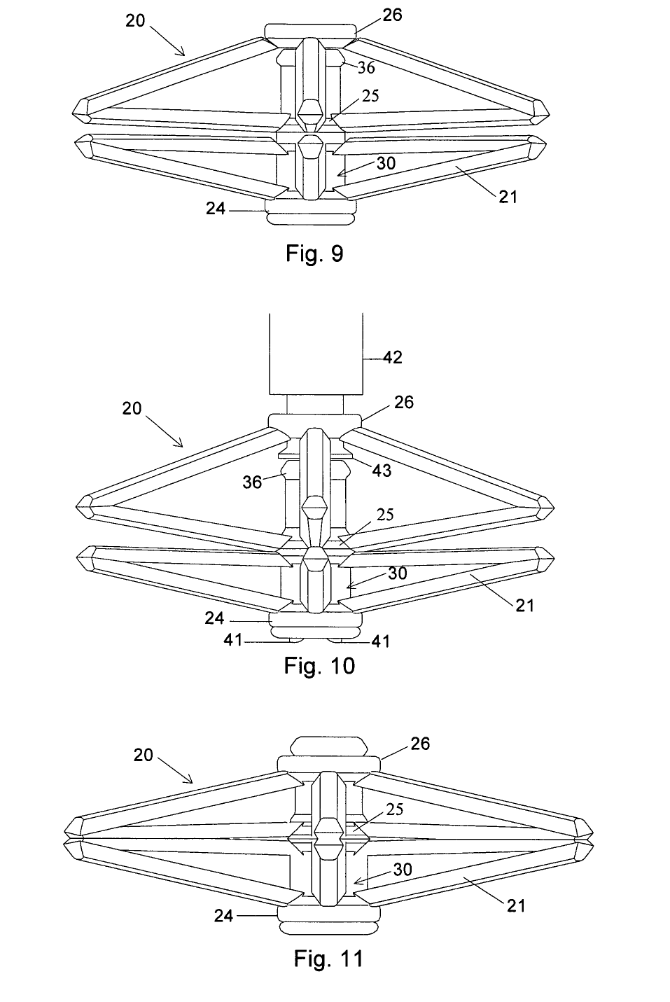

In FIG. 6, the closure device 20 of FIG. 5 is depicted in a semi-expanded positioning configuration, in which the distal and proximal end portions 24, 26 of the closure device 20 have been moved towards the central portion 25. The hinge sections 27, 28 of the first and second struts 22, 23 have thereby been forced to move outwards from the central axis of the closure device 20, and the articulated arms 22a, 22b and 23a, 23b have assumed an angled relation to the central axis of the closure device 20. The semi-expanded configuration shown in FIG. 6 can be used to determine the proper position for the closure device 20, and can also be regarded as a positioning configuration prior to a closed configuration described below in conjunction with FIG. 9 or FIG. 10. The positioning configuration is defined as all intermediate states between the introduction configuration defined above and the closed configuration, which will be further described and defined below. Another example of a positioning configuration is illustrated in FIG. 7.

As can be seen in FIG. 6, the closure device 20 comprises further a locking member 30, which is separately illustrated in FIG. 8. The locking member 30, which according to the invention may constitute a separate part of closure device 20, or may be an integrated part thereof, comprises a hollow body 31, which along is length is provided with several portions with different outer diameters. More specifically, the body 31 of the locking member 30 comprises a distal end rim 32, a distal portion 33, an intermediate portion 34, a proximal portion 35, and a proximal end rim 36. The distance between the distal end rim 32 and the proximal end rim 36 is considerably smaller than the length of the tubular member 21. As the observant reader already may have appreciated, the respective outer diameters of the body 31 of the locking member 30 are related to the respective diameters of the tubular member 21 of the closure device 20. Thus, the diameter of the distal end rim 32 is larger than the inner diameter of the distal end portion 24 of the tubular member 20, while the inner diameter of the distal end portion 24 is larger than the other diameters of the body 31 of the locking member 30, such that the distal end portion 24 of the tubular member 21 can slide over the locking member 30 until the distal end portion 24 abuts the distal end rim 32. The outer diameter of the distal portion 33 of the locking member 30 is adapted to the inner diameter of distal end portion 24 of the tubular member 21, while the diameter of the intermediate portion 34 is adapted to the diameter of the central portion 25 of the tubular member 21. The inner diameter of the proximal end portion 26 of the tubular member 21 is adapted to the outer diameter of the proximal portion 35 of the locking member 30, and is slightly less than the diameter of the proximal end rim 36. During use, the proximal end portion 26 of the tubular member 21, which is made from a somewhat elastic material, must therefore be forced over the proximal end rim 36 and can then slide on the proximal portion 35. As can be seen in FIG. 8, the locking member 30 comprises preferably a recess 37, which provides the proximal end rim 36 with a certain resilience which facilitates the sliding of the proximal end portion 26 of the closure device 20 over the proximal end rim 36 of the locking member 30.

As indicated above, the closure device 20 can assume an infinite number of positioning configurations during a positioning operation in which a septal defect is located and the closure device 20 is positioned therein. According to the present invention, there is, however, a well-defined endpoint for the positioning operation. This endpoint, which is referred to as the closed configuration of the closure device 20, is illustrated in FIG. 9, where it can be seen that the central portion 25 of the tubular member 21 has been positioned over the intermediate portion of the locking member 30, while the proximal end portion 26 of the tubular member 21 abuts the proximal end rim 36 of the locking member 30. (For illustrative purposes only, there is a small gap between the proximal end portion 26 and the proximal end rim 36 in FIG. 9.) As has been mentioned above, the inner diameter of the proximal end portion 26 is slightly less than the diameter of the proximal end rim 36, which means that further compression of the tubular member 21 is not possible—unless an extra operation is carried out such that the proximal end portion 26 is forced over the proximal end rim 36. The closed configuration of FIG. 9 thereby constitutes a well-defined state.

The situation illustrated in FIG. 9 is, however, only one example of a closed configuration. In practice, the movements of the closure device are effectuated by the previously mentioned mechanical actuator, parts of an example of which are illustrated in FIG. 10 together with the tubular member 21 as well as the locking member 30. The mechanical actuator comprises a holder and an actuating member. The holder comprises a holder member 41 and a locking pin (not seen in FIG. 10), while the actuating member comprises a pusher 42 and a retractor 43 (in the related applications referred to herein this element is called an actuating member). By moving the actuating member back and forth, a doctor can during a preceding positioning operation let the tubular member 21 assume different positioning configurations, to thereby locate a septal defect (or some other type of tissue opening, e.g. a percutaneous puncture in an artery wall) and position the closure device 20 in the opening of the defect. The terms “actuating member” and “holder member” should not be interpreted too literally; it is actually the relative motion between these two members that is important. In other words, seen from some fixed position it could actually be the holder that is moved, whereas the actuating member is still. Further, during the first three stages, i.e. the introduction, positioning and closed configuration of the closure device 20, the pusher 42 and the retractor 43 move together as a first unit, while the holder member 41 and the locking pin move together as a second unit. In the situation illustrated in FIG. 10, the distal end of the retractor 43 abuts the proximal end rim 36 of the locking member 30. (For illustrative purposes only, there is a small gap between the distal end of the retractor 43 and the proximal end rim 36 in FIG. 10.) Thus, FIG. 10 illustrates a well-defined end position for the positioning operation, in which no further compression of the tubular member 21 is possible by maneuvering of the actuating member in relation to the holder without forcing proximal end portion 26 over proximal end rim 36. If, on the other hand, an actuating member were engaged inside a proximal end portion of a tubular member, the situation would resemble the situation illustrated in FIG. 10, i.e. a well-defined end point of the positioning operation—in which no further compression of the tubular member is possible without forcing an end portion over an end rim—would be when a proximal end portion of the tubular member abuts a proximal end rim of a locking member. The closed configuration of a closure device according to the present invention is thereby defined as the extreme end position of the positioning configurations, wherein an end portion of a locking member prevents further compression of a tubular member. This definition also encompasses closure devices where a proximal end portion of a locking member prevents further compression of a tubular member, i.e. a closure device where a distal end portion of a tubular member is pulled over an enlarged distal end rim of a locking member rather than—as in the closure device described above—having a proximal end portion of a tubular member that is pushed over an enlarged proximal end rim of a locking member.

From FIG. 10 it may be realized that when the retractor 43 abuts the proximal end rim 36 of the locking member 30, the closure device 20 can be transferred into the final locked state by movement of the pusher 42. To accomplish this, the pusher 42 (which can slide with respect to the retractor 43) is advanced, so that the proximal end portion 26 of the tubular member 21 is forced up and over the proximal end rim 36 of the locking member 30. This movement requires that the proximal end portion 26 and/or the proximal end rim 36 possesses a certain degree of resilience.

The final locked configuration of the closure device 20 is illustrated in FIG. 11, wherein the distal and proximal end portions 24, 26 of the tubular member 21 have been fully moved towards each other until the central portion 25 of the tubular member 21 is positioned over the intermediate portion of the locking member 30, and the proximal end portion 26 of the tubular member 21 has been moved over the proximal end rim 36 of the locking member 30. The closure device 20 is held in this compressed state due to the enlarged distal and proximal end rims 32, 36 of the locking member 30, which have diameters larger than the distal end portion 24 and the proximal end portion 26, respectively. The closure device 20 can then be released and left in this locked configuration by maneuvering of the holder member and locking pin mentioned above. The locked configuration of a closure device is thereby defined as the configuration in which the closure device is fully expanded, and in which the closure device maintains its configuration without assistance of a mechanical actuator.

The septal defect closure device has been shown with proximal and distal struts having equal lengths. It is, however, possible to provide a closure device having proximal struts with one length and distal struts with a different length. It may, for example, be desirable to arrange a closure device in such a way that the left part of the closure device, i.e. the part that is implanted into the left atrium of a heart, is smaller than the right part of the closure device, to thereby reduce the amount of artificial material introduced into the left atrium, which in turn may reduce the formation of thrombogenic material therein. In this context, it should be recognized that it is not mandatory that a heart is accessed via the venous system, as is shown in FIGS. 2 to 5, but the heart could be accessed via the arterial side. This means that if a doctor wishes to place a smaller part of a closure device at the left side of a heart than at the right side of the heart, then this smaller part (i.e. the shorter struts) will constitute the distal set of struts if the heart is accessed via the venous system, whereas the smaller part will constitute the proximal set of struts if the heart is accessed through the arterial system. It can therefore be appreciated that it can be advantageous to provide a closure device in the form of two separate tubular members (and a separate locking member) as this would provide a doctor with the possibility to tailor a septal defect closure device to the specific medical situation at hand, without the necessity of producing an excessive large number of closure devices with different dimensions.

It has already been mentioned that the length of the distal struts can differ from the length of the proximal struts; and it is also possible to have different lengths of the articulated arms within a strut set, such that, for example, the distal arms are longer than the proximal arms, or vice versa. The arms that actually contact a septum or a vessel wall can, for example, be shorter than the arms that do not contact the septum or the vessel wall, to thereby ensure a reliable closing of a septal defect in the septum or a puncture hole in the vessel wall.

It has already been mentioned that a locking member can constitute a separate part of a closure device, and a locking member can be made from a first material and a tubular member can be made from a second material. With different materials some specific advantages can be achieved. If, for example, the closure device is a resorbable closure device, then the resorption time of the material in the locking member can be different from the resorption time of the material in the tubular member, such that the locking force between the two members during the degradation of the closure device is reduced and ultimately lost in a controllable and predictable way. In this respect it may be advantageous if the material of the tubular member has a shorter resorption time than the material of the locking member. Further, whether or not the materials are resorbable materials, different requirements are put on the different pieces. For example, the material in the hinge portions of a tubular member must be flexible and have a high tenacity, whereas the locking member must have a rather high stiffness. Also in a resorbable closure device it can be necessary to have one material in a locking member and another material in a tubular member, because of the different dimensions involved. It can, for example, be necessary to have a material with a relatively long resorption time in the thin hinge portions of the tubular member in order to match the resorption time of the material in a thick-walled locking member.

Examples of resorbable materials for the tubular member and the locking member may include, but are not limited to, those materials made from aliphatic polyesters, polyether esters, and polycarbonates. More specifically, synthetic resorbable polymers such as homopolymers and copolymers made from any of the monomers lactide, glycolide, epsilon-caprolactone, trimethylene carbonate, and paradioxanone are advantageous because of their long clinical use.

The tubular member could preferably be made from a semi-crystalline material with a lower tensile modulus than the locking member. As previously stated, it could, e.g. because of the hinge portions, be an advantage to have a more flexible material in the tubular member. Such material is preferably made from a block copolymer characterized by having a soft middle part characterized by having a glass transition temperature below room temperature and a semi-crystalline part at each end of the soft middle part. The semi-crystalline part could be polymerized from any of the monomers glycolide, lactide, or paradioxanone. Since polyparadioxanone is a relatively soft and pliable material compared to polyglycolide and polylactide, the tubular member can be made from pure polyparadioxanone itself.

The locking member can be made from any of the above materials, but to secure the locking mechanism it is advantageous if the material is stiffer than the material used in the tubular member. The material should also preferably resorb at a somewhat slower pace than the tubular member. The locking member could also be made from amorphous or semi-crystalline material, and preferably from homopolymers or copolymers where the main monomer component is lactide, caprolactone, or paradioxanone.

A particular advantage of the groups of synthetic resorbable polymers mentioned above is that various mechanical properties can be accomplished by simply changing the monomer composition in the homopolymer or copolymer. Further, in contrast to natural biopolymers, these materials can be molded and machined into complex structures, and by varying the monomer composition large time spans can be achieved for their resorption times.

It may be appreciated that it can be advantageous to provide a radiopaque closure device which is visible in an X-ray machine. When the closure device is made from a synthetic resorbable polymer, a radiopaque closure device can conveniently be produced by mixing the polymer with suitable radiopaque agent. A suitable radiopaque agent is barium sulfate, which can be blended into the polymer or copolymer in an amount between 5% and 50%, and more preferably in an amount of 15% to 30%, to obtain the opacity needed in order to locate the closure device during an X-ray observation. Radiopaque materials can be used in a tubular member of the closure device, but is preferably used in the locking member, which marks the centre of the device. The radiopaque agent, e.g. barium sulfate, can—instead of being mixed with the polymer—be introduced into preformed holes in the closure device, which are then sealed by a synthetic resorbable material. As an alternative, preformed holes can be plugged with a resorbable material containing a large amount of a radiopaque agent, e.g. barium sulfate.

FIG. 12 illustrates a medical apparatus 100 comprising a mechanical insertion and delivery assembly 101, a closure device 102, and a delivering catheter 103. The closure device 102 is of the same general design that has been described above, and comprises an elongated tubular member 121 and a locking member 130. The tubular member 121 comprises a first or distal set of struts 122, which extend from a first or distal portion 124 of the tubular member 121 to a central portion 125 thereof. The tubular member 121 comprises further a second or proximal set of struts 123, which extend from the central portion 125 to a second or proximal portion 126 of the tubular member 121. The locking member 130 comprises a hollow body 131, which, in turn, comprises a distal end rim 132, a distal portion 133, an intermediate portion 134, a proximal portion 135, and a proximal end rim 136. The body 131 of the locking member 130 is generally hollow, except for its most distal portion which is solid to avoid leakage of blood through the locking member 130. The insertion assembly 101 comprises a housing 104, a coil spring 105, a holder 106, and an actuator 107. The holder 106 is comprised of a locking pin 141, a locking pin handle 142, a tubular holder member 143, and a holder handle 144. The actuator 107 comprises a tubular retractor 145, a tubular pusher 146, and an actuator handle 147. Both the pusher 146 and the actuator handle 147 are integrated parts of the housing 104, while the retractor 145 is slidably arranged inside the tubular pusher 146. The coil spring 105 is mounted in the housing 104, with a distal end of the coil spring 105 being supported by an internal support member 148 in the housing 104 and a proximal end of the coil spring 105 being supported by the holder handle 144, such that the coil spring 105 strives to increase the distance between the holder handle 144 and the actuator handle 147. The distal end of the tubular holder member 143 is cut and bent outwards into two grip members 149, which are engaged in a recess or cavity in the interior of the hollow body 131 of the locking member 130. The grip members 149 are prevented from approaching each other, i.e. being squeezed together, by the locking pin 141, which in this stage of a delivering operation is disposed within the tubular holder member 143. The distal end of the tubular retractor 145 is upended; and the proximal portion 126 of the tubular member 121 is fixedly arranged between the upended distal end of the retractor 145 and the distal end of the pusher 146. The distal portion 124 of the tubular member 121 has been threaded onto the hollow body 131 of the locking member 130, and fits snugly over the distal portion 133 and abuts the distal end rim 132 of the locking member 130. As will be further elucidated below, except for in the penultimate stage of a delivering operation, the retractor 145 and the pusher 146 move together as a unit in response to reciprocal movements of the actuator handle 147 in relation to the holder handle 144. Since the tubular member 121 is fixated at the actuator 107 at one end and at the distal portion 133 of the locking member 130 at the other end, the corresponding relative motion between the holder 106 and the actuator 107 causes the tubular member 121 to compress or expand longitudinally.

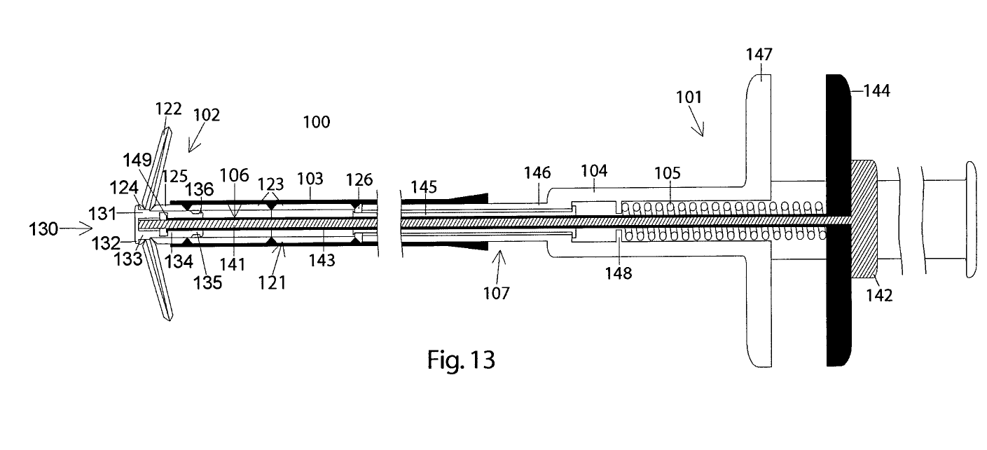

In FIG. 12, the medical apparatus 100 is shown in an introduction phase, in which a distal portion of the tubular member 121 has been advanced out of the delivering catheter 103, whereas the more proximal portion of the tubular member 121 still is within the delivering catheter 103. It can further be noted that the holder handle 144 and the actuator handle 147 abut each other, which corresponds to maximal compression of the coil spring 105. To proceed from the introduction configuration of FIG. 12 to a positioning configuration as described above, the actuator handle 147 is advanced in relation to the holder handle 144, which means that the actuator 107 moves relative to the holder 106. More specifically, this relative movement causes the distal ends of the retractor 145 and pusher 146 to come closer to the distal end of the holder member 143; and, because the tubular member 121 is fixated between the holder 106 and the actuating member 107, the distal struts 122 of the tubular member 121 move outwards and assume an essentially perpendicular angle to the longitudinal central axis of the closure device 102, as has been described above. This stage of a delivering operation is illustrated in FIG. 13.

FIG. 13 shows the medical apparatus 100 in a positioning configuration, in which the distal struts 122 of the tubular member 121 have been expanded in response to a relative motion between the holder 106 and the actuator 107. In this exemplifying embodiment of the present invention, the positioning configuration of FIG. 13 has been effectuated by the coil spring 105 in that when the distal struts 122 of the tubular member 121 are advanced out of the delivering catheter 103, the action of the coil spring 105 separates the holder handle 144 from the actuator handle 147, which reduces the distance between the grip members 149 at the distal end of the holder member 143 and the proximal portion 126 (held between the upended distal end of the retractor 145 and the pusher 146), and causes the distal struts 122 to assume an essentially perpendicular angle to the central axis of the closure device 102. It should, however, be understood that the force that separates the holder handle 144 from the actuator handle 147 could have been provided manually, i.e. without the use of a coil spring 105. It should further be clear that the terms “holder” and “actuator”, and terms that are derived therefrom, e.g. “holder member”, “holder handle” and “actuator handle”, should not be given any particular inherent meaning as far as it comes to their corresponding movements. It is the relative motion between the holder 106 and the actuator 107, and—to be described below—between other parts, e.g. retractor 145 and pusher 146, of the insertion assembly 101 that accomplishes the desired effect on the closure device 102.

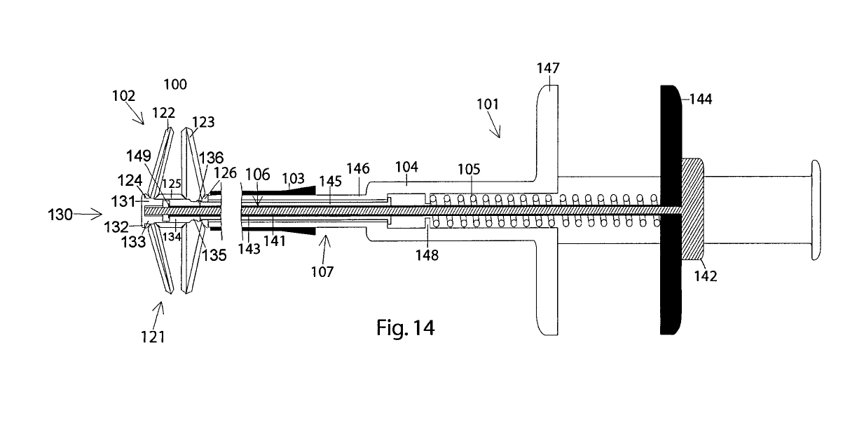

As already has been discussed in conjunction with FIG. 3, the positioning configuration illustrated in FIG. 13 is used to locate a tissue wall, e.g. a septum in a heart, or a vessel wall, and to position the distal struts 122 of the tubular member 121 in contact with the distal surface of this tissue wall. This is accomplished by retracting the insertion assembly 101 until the distal struts 122 abut the distal surface of the tissue wall. The delivering catheter 103 is subsequently retracted relative to the insertion assembly 101 such that also the proximal struts 123 of the tubular member 121 are outside the distal end of the delivering catheter 103; and the closure device 102 is compressed such that the tissue wall is sandwiched between the distal struts 122 and the proximal struts 123 of the tubular member 121. The closed configuration of the closure device 102 is depicted in FIG. 14, which shows that the distance between the holder handle 144 and the actuator handle 147 has been further increased, which, in turn, means that the distance between the distal end of the holder member 143 and the distal end of the pusher 146 has been reduced and caused both the distal struts 122 and proximal struts 123 to assume an essentially perpendicular angle to the central axis of the closure device 102. In this embodiment of the invention also this movement is accomplished by the force exerted by the coil spring 105, but it is to be understood that the same movement could be accomplished manually by a doctor or other trained medical personnel. In FIG. 14 it should in particular be noted that the closure device 102 still is fixedly arranged between the holder 106 and the actuator 107, which means that the whole procedure can be reversed. In other words, by pressing together the holder handle 144 and the actuator handle 147, the closure device 102 can again be transformed to the delivering configuration illustrated in FIG. 12, and the whole medical apparatus 100 could be retracted out of a patient's body and be disposed, or the steps shown in FIGS. 12, 13 and 14 could be repeated in order to reposition the closure device 102 in an opening or defect in an organ.

If a doctor determines that the closure device 102 is correctly positioned in an opening or defect in an organ, and, consequently, that the delivering operation described in conjunction with FIGS. 12, 13 and 14 has been successful, he or she may decide to irreversibly leave the closure device 102 in the organ, to close the opening or defect therein. To this effect, the closure device 102 has to be transferred from the closed configuration of FIG. 14 to a locked configuration. Here it should be noted that in FIG. 14 the distal end of the retractor 145 abuts the proximal end rim 136 of the locking member 130; and, since the inner diameter of the tubular retractor 145 is smaller than the diameter of the proximal end rim 136 of the locking member 130, the retractor 145 cannot be advanced any further in relation to the grip members 149 at the distal end of the holder member 143.

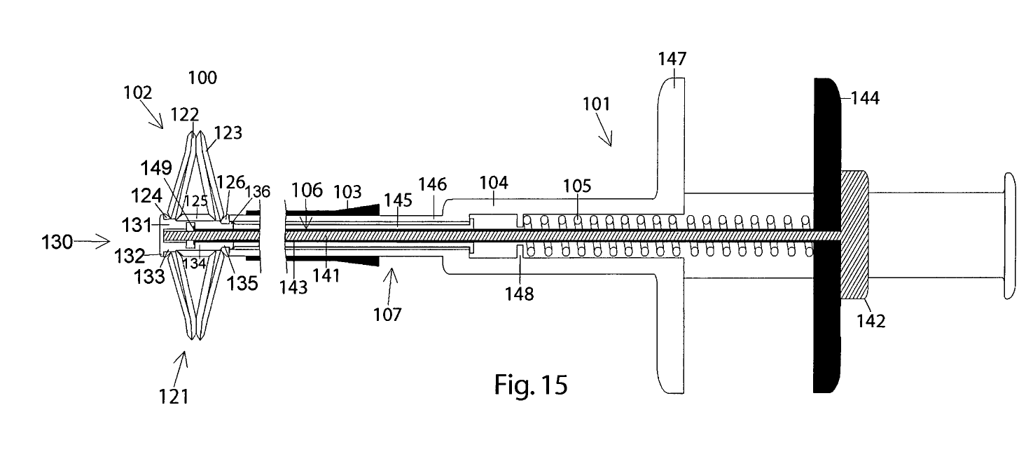

FIG. 15 illustrates the medical apparatus 100 in a configuration in which the closure device 102 is irreversibly locked. By comparing FIG. 14 and FIG. 15 it can be noted that the distance between the holder handle 144 and the actuator handle 147 is slightly larger in the locked configuration of FIG. 15 than in the closed configuration of FIG. 14, which means that the pusher 146 has been moved closer to the distal end of the holder member 143. As was explained above, the retractor 145, which is slidably arranged in the tubular pusher 146, can, however, not be moved any closer to the distal end of the holder member 143, resulting in the pusher 146 pushing the proximal portion 126 of the tubular member 121 over the proximal end rim 136 of the locking member 130. In this stage of a delivering operation, the retractor 145 and the pusher 146 no longer move in common as a unit, instead it is the relative motion between the pusher 146 and the retractor 145 that accomplishes the transition from the closed configuration of FIG. 14 to the locked configuration shown in FIG. 15. A special feature of the illustrated embodiment of the present invention is that the spring force exerted by the coil spring 105 is too small to cause the retractor 145 to move relative to the pusher 146. The locked configuration is instead achieved by a user who manually advances the actuator handle 147 relative to the holder handle 144. It may be appreciated that this feature is particularly advantageous, because—as has been emphasized before—the closed configuration represents a reversible state from which the closure device 102 can be reversibly operated by the mechanical insertion and delivery assembly 101, whereas the locked configuration is a state from which the closure device 102 cannot be retrieved from a patient's body, at least not without an extensive medical intervention, and the possibility of an unintentional locking of the closure device 102 should therefore be avoided.

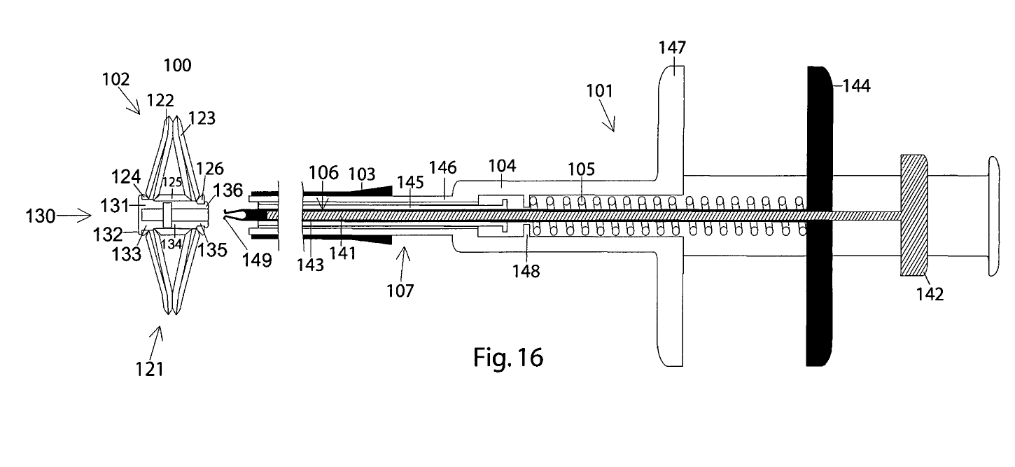

The last important operation that a doctor carries out is to release the closure device 102 from the insertion assembly or tool 101. This is done by retracting the locking pin handle 142 in relation to the holder handle 144, i.e. the locking pin 141 is moved relative to the holder member 143. More specifically, by retracting the locking pin handle 142 such that the locking pin 141 is withdrawn proximally of the grip members 149 at the distal end of the holder member 143, the grip members 149 are free to approach each other; and, in response to a retracting movement of the mechanical insertion tool 101, the grip members 149 are disengaged from the recess or cavity in the interior of the hollow body 131 of the locking member 130. FIG. 16 illustrates how the closure device 102 is released from the mechanical insertion assembly 101, and illustrates further that the grip members 149 were preformed with a tendency to point towards each other. Optionally, an insertion assembly, like insertion assembly 101, may be provided with a safety mechanism that prevents the withdrawal of a locking pin before an actuator handle has been moved to its outmost position with respect to a holder handle, to avoid the risk of unintentionally disengaging a closure device from an insertion assembly.

Other aspects, features, variations, and ways of using the present invention are described in the U.S. patent applications entitled “Closure Device” and filed under attorney docket numbers 030481/0249; 030481/0250; and 030481/0254 concurrently herewith. The entire contents of these related applications are incorporated herein by reference. Features in these different applications may be combined with each other.

Although the present invention has been described with reference to specific embodiments, also shown in the appended drawings, it will be apparent for those skilled in the art that many variations and modifications can be done within the scope of the invention as described in the specification and defined with reference to the claims below. It may in particular be appreciated that a holder, which herein has been described as comprising two members, a holder member and a locking pin, can be in the form of a single member, which then is releasably engaged in a locking member, e.g. threaded or keyed into a locking member. Similarly, an actuation assembly, which herein also has been described as comprising two members, a retractor and a holder, can be in the form of a single member, which then is releasably engaged in a proximal portion of a tubular member, e.g. threaded or keyed into a tubular member. It may also be pointed out that the mechanical insertion and delivery assembly or tool described herein could, with minor modifications, be used to operate a closure device that is of a unitary construction. In fact, the locking member described above could be regarded as part of a holder to releasably engage the holder in a distal portion of a tubular member. An insertion assembly comprising a spring that effectuates a relative movement between two members could also be used in combination with other types of closure devices than the closure device described herein, and in particular in combination with a closure device that comprises an expandable distal portion and an expandable proximal portion, which are designed to be positioned on each side of a tissue wall. As has been explained above, it is especially advantageous if the spring force of the spring is adapted to the closure device such that the spring can accomplish a closed, reversible configuration of the closure device, but cannot accomplish a locked, non-reversible configuration of said closure device. Other parts, in particular a locking pin and a holder member, of an insertion assembly could also be used in combination with other types of closure devices, and in particular closure devices comprising an expandable distal portion and an expandable proximal portion.

What is claimed is:

1. A medical apparatus comprising:
a closure device having a longitudinal central axis and comprising a tubular member having a length and an expandable distal portion extending between a distal end portion and a central portion and an expandable proximal portion extending between said central portion and a proximal end portion,
an insertion assembly comprising a holder and an actuator, the holder being releasably engaged in a locking member and the actuator being releasably engaged with the proximal end portion,
and, in response to a relative movement between the holder and the actuator, the closure device is movable between a first elongated tubular introduction configuration and a second positioning configuration in which the distal and proximal end portions have been moved towards each other such that said expandable distal and/or proximal portions have expanded radially away from said longitudinal central axis, and
wherein the locking member is a separate locking member having a distal end rim with a diameter larger than the diameter of the distal end portion and a proximal end rim with a diameter larger than the diameter of the proximal end portion, the distance between the distal and proximal end rims being smaller than the length of the tubular member and the locking member being positioned inside the tubular member such that the distal end rim abuts the distal end portion.

2. A medical apparatus according to claim 1, wherein said expandable distal portion comprises a set of struts provided with a hinge section that can act as a hinge, and, in the second positioning configuration, the hinge sections have moved radially away from said longitudinal central axis.

3. A medical apparatus according to claim 1, wherein said expandable proximal portion comprises a set of struts provided with a hinge section that can act as a hinge, and, in the second positioning configuration, the hinge sections have moved radially away from said longitudinal central axis.

4. A medical apparatus according to claim 1, wherein the actuator comprises a pusher and a retractor, the distal end of which is upended, and wherein the proximal end portion of the tubular member is fixated between the upended distal end of the retractor and the distal end of the pusher.

5. A medical apparatus according to claim 4, wherein the retractor is slidably arranged inside the pusher, such that, when the pusher is moved closer to the distal end of the holder and the retractor abuts the proximal end rim of the locking member, the pusher pushes the proximal end portion of the tubular member over the proximal end rim of the locking member.

6. A medical apparatus comprising:
a closure device comprising an expandable distal portion extending between a distal end portion and a central portion and an expandable proximal portion extending between said central portion and a proximal end portion, and
an insertion assembly comprising a holder and an actuator, the holder being releasably coupled to the distal end portion and the actuator being releasably coupled to the proximal end portion,
wherein the insertion assembly further comprises a spring which effectuates a relative movement between the holder and the actuator, which relative movement causes the closure device to move from a first elongated introduction configuration to a second positioning configuration in which the distal and proximal end portions have been moved towards each other such that said expandable distal and/or proximal portions have expanded.

7. A medical apparatus according to claim 6, wherein a spring force of the spring is too small to place the closure device in a locked configuration.

8. A medical apparatus comprising:
a closure device comprising an expandable distal portion extending between a distal end portion and a central portion and an expandable proximal portion extending between said central portion and a proximal end portion, and
an insertion assembly comprising a holder and an actuator, the holder being releasably coupled to the distal end portion and the actuator being releasably coupled to the proximal end portion,
and, in response to a relative movement between the holder and the actuator, the closure device is movable between a first elongated introduction configuration and a second positioning configuration in which the distal and proximal end portions have been moved towards each other such that said expandable distal and/or proximal portions have expanded, and
wherein the holder comprises a locking pin and a tubular holder member having a distal end provided with grip members, and the locking pin is adapted to be disposed inside the tubular holder member, to prevent the grip members from approaching each other.
